(19) United States
(12) Patent Application Publication
(10) Pub. No.: US 2024/0180929 A1
(43) Pub. Date: Jun. 6, 2024

(54) TREATMENT OF INFECTIOUS DISEASES USING BCL-2 FAMILY PROTEIN INHIBITORS

(71) Applicant: TEXAS BIOMEDICAL RESEARCH INSTITUTE, San Antonio, TX (US)

(72) Inventors: Larry Schlesinger, San Antonio, TX (US); Eusondia Arnett, San Antonio, TX (US)

(21) Appl. No.: 18/042,648

(22) PCT Filed: Aug. 23, 2021
(86) PCT No.: PCT/US2021/047074

§ 371 Date: Feb. 23, 2023

Related U.S. Application Data
(60) Provisional application No. 63/069,086, filed on Aug. 23, 2020.

(51) Int. Cl. A61K 31/635; A61K 31/4162; A61K 31/426; A61K 31/519; A61K 31/5377; A61K 31/553; A61K 45/06; A61P 31/06

(52) U.S. Cl. CPC A61K 31/635; A61K 31/4162; A61K 31/426; A61K 31/519; A61K 31/5377; A61K 31/553; A61K 45/06; A61P 31/06

(57) ABSTRACT

Provided are methods of treating infectious diseases by administering a BCL-2 inhibitor and a MCL-1 inhibitor to a subject in need thereof. One such infectious disease is an infection of Mycobacterium tuberculosis. Also provided are methods of inhibiting the growth of Mycobacterium tuberculosis in a granuloma by contacting the granuloma with a BCL-2 inhibitor and a MCL-1 inhibitor.

### CROSS-REFERENCE TO RELATED APPLICATION

This application is a U.S. National Phase Under 35 U.S.C. § 371 of International Application No. PCT/US2021/047074, titled “TREATMENT OF INFECTIOUS DISEASES USING BCL-2 FAMILY PROTEIN INHIBITORS,” filed Aug. 23, 2021, which is a PCT application claiming priority to and the benefit of U.S. Provisional Application No. 63/069,086, titled “TREATMENT OF INFECTIOUS DISEASES USING BCL-2 FAMILY PROTEIN INHIBITORS,” filed Aug. 23, 2020. The entire contents of each of the above applications are hereby incorporated by reference.

### GOVERNMENT SUPPORT

This invention was made with Government support under Federal Grant no. 1R01AI136831-01AI awarded by the National Institutes of Health. The Federal Government has certain rights to this invention.

### TECHNICAL FIELD

This disclosure relates to the treatment of infectious diseases through the use of combinations of inhibitors of proteins that regulate cell death.

### BACKGROUND

Antimicrobial resistance is a significant and growing problem for treatment of many infectious diseases, including tuberculosis (TB), leading to a marked increase in human suffering and death in the US and worldwide. The Center for Disease Control (CDC) estimates that people in the United States contend with more than 2.8 million antibiotic-resistant infections a year—and more than 35,000 die as a result. In 2018, there were over 700 incidences of drug resistant TB and over 600 TB related deaths in the US. Worldwide, in 2018 there were about half a million incidences of drug resistant TB and 1.4 million deaths. Current treatments for TB involve administering a cocktail of antibiotics for at least 6 months.

### SUMMARY

Disclosed herein are compositions and methods addressing the shortcomings of the art, and may provide any number of additional or alternative advantages. The combination of using an antibiotic and a host-directed therapeutic (HDT, bolstering the immune response) holds promise for overriding drug resistant infections. Provided here are methods of treating infections caused by Mycobacterium tuberculosis (M.tb) and also methods for treating infections caused by other infectious pathogens, such as non-tuberculous mycobacteria, histoplasma, coccidioides, and blastomyces. One such method includes administering to a subject in need thereof a BCL-2 inhibitor and a MCL-1 inhibitor. The BCL-2 inhibitor includes venetoclax (ABT-199), ABT-263, S55746, ABT-737, or any combinations thereof. The MCL-1 inhibitor includes S63845, A-1210477, MIM-1, MIK665/S-64315, 483-LM, AZD5991, AMG-176, AMG-397, UMI-77, VU661013, JKY-5-037, or any combinations thereof. One such method includes administering to a subject in need thereof a combination of venetoclax as a BCL-2 inhibitor and S63845 as the MCL-1 inhibitor. Another such method includes administering to a subject in need thereof a combination of venetoclax as a BCL-2 inhibitor and MIK665/S-64315 as the MCL-1 inhibitor. Another such method includes administering to a subject in need thereof a combination of venetoclax as a BCL-2 inhibitor and AZD5991 as the MCL-1 inhibitor.

Provided here are methods of inhibiting the growth of M.tb in a host organ granuloma, a unique tissue immune response environment that walls off M.tb and promotes bacterial resistance. One such method includes contacting the granuloma with a BCL-2 inhibitor and a MCL-1 inhibitor. The BCL-2 inhibitor includes venetoclax (ABT-199), ABT-263, S55746, ABT-737, or any combinations thereof. The MCL-1 inhibitor includes S63845, A-1210477, MIM-1, MIK665/S-64315, 483-LM, AZD5991, AMG-176, AMG-397, UMI-77, VU661013, JKY-5-037, or any combinations thereof. One such method includes administering to a subject in need thereof a combination of venetoclax as a BCL-2 inhibitor and S63845 as the MCL-1 inhibitor. Another such method includes administering to a subject in need thereof a combination of venetoclax as a BCL-2 inhibitor and MTK665/S-64315 as the MCL-1 inhibitor. Another such method includes administering to a subject in need thereof a combination of venetoclax as a BCL-2 inhibitor and AZD5991 as the MCL-1 inhibitor.

The subject of these methods of treatment may have active tuberculosis. The subject of these methods of treatment may have latent tuberculosis. In an embodiment, the method includes administering to the subject one or more of a BCL-2 family protein inhibitor (e.g., a MCL-1 inhibitor and a BCL-2 inhibitor) along with a therapeutically effective amount of one or more of anti-tuberculosis antibiotics. Examples of anti-tuberculosis antibiotics include first-line compounds, such as isoniazid, rifampin, rifabutin, rifapentine, ethambutol, and pyrazinamide. Other examples of anti-tuberculosis antibiotics include second-line compounds, such as cycloserine, capreomycin, kanamycin, amikacin, fluoroquinolones (e.g. levofloxacin and moxifloxacin), ethionamide, protionamide, terizidone, and para-aminosalicylic acid. In an embodiment, the BCL-2 family protein inhibitors (e.g., a MCL-1 inhibitor and a BCL-2 inhibitor) are administered to treat a subject having TB in addition to other co-morbidities, such as other infectious or metabolic diseases. In an embodiment, the infectious disease is caused by HIV.

Numerous other aspects, features and benefits of the present disclosure may be made apparent from the following detailed description taken together with the figures. The pharmaceutical compositions can include compositions described herein along with other components, or ingredients depending on desired prevention and treatment goals. It should be further understood that both the foregoing general description and the following detailed description are exemplary and explanatory and are intended to provide further explanation of the invention as claimed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. Embodiments are illustrated by way of example and not by way of limitation in the figures of the accompanying drawings.

FIG. 1A is an image of a western blot showing an increase in MCL-1 following M.tb infection in bone marrow-derived macrophages (BMDMs). FIGS. 1B and 1C are graphical representations of the results of a CFU (colony forming unit) assay showing that treating murine BMDM or lung macrophages infected with M.tb with a combination of S63845 and ABT-199 resulted in a reduction in the number of M.tb CFUs comparable to that achieved with pan BCL-2 family inhibitors sabutoclax and TW37. FIG. 1B sets forth CFUs normalized based on the solvent/DMSO control in BMDMs, and FIG. 1C sets forth the absolute number of CFUs in lung macrophages.

FIG. 2A is a graphical representation of the results of a CFU assay showing that treating human granuloma structures infected with M.tb with a combination of 10 μM S63845 and 10 μM ABT-199 resulted in reduced M.tb growth as compared to treatment with MCL-1 or BCL-2 inhibitors alone. FIGS. 2B and 2C are graphical representations of the results of a CFU assay showing that treating human granulomas infected with M.tb with a combination of 10 μM ABT-199 and 10 μM S63845 reduced M.tb growth in granulomas to a similar extent as the general BCL-2 family inhibitor-sabutoclax at 7 days (FIG. 2C) as compared to treatment with MCL-1 or BCL-2 inhibitors alone, or combinatorial treatment with BCL-2 inhibitor ABT-199 and select other MCL-1 inhibitors with reduced activity relative to the MCL-1 inhibitor S63845.

FIGS. 3A-3C are graphical representations of the data from a luciferase-based growth assay showing human monocyte-derived macrophages (MDMs) treated with the general BCL-2 family inhibitor sabutoclax (FIG. 3A), ABT-199 (FIG. 3B), and S63845 (FIG. 3C). Treating human monocyte-derived macrophages (MDMs) with 10 μM S63845 (FIG. 3C) reduced M.tb growth after 4 days, but to a lesser extent than the general BCL-2 family inhibitor sabutoclax (FIG. 3A). FIGS. 3D-3F are graphical representations of the data from a luciferase-based growth assay using MDMs from a different donor showing MDMs treated with the general BCL-2 family inhibitor sabutoclax (FIG. 3D), ABT-199 (FIG. 3E), and S63845 (FIG. 3F). There was reduced growth of M.tb in MDMs after (i) 7 days of treatment with 1 μM S63845 (FIG. 3F) and (ii) 6 days of treatment with 10 μM ABT-199 (FIG. 3E).

FIGS. 4A and 4B are graphical representations of the data from a luciferase-based growth assay showing reduced M.tb growth following 4 days of treatment with a combination of ABT-199 and S63845 using MDMs from two different donors.

FIG. 5 is a microscope image showing HIV present in a M.tb/HIV co-infected in vitro granuloma. HIV is stained in green, and the human cells in the granuloma are stained in blue.

FIGS. 6A and 6B are graphical representations of the CFU assay using lungs and spleen from mice infected with M.tb and treated with a combination of S63845 and ABT-199, along with the controls.

FIGS. 6C-6F and 6G-6J are photographic images of immunohistochemistry of murine lungs subject to acid fast staining and hematoxylin and eosin staining, respectively, from mice infected with M.tb and treated with a combination of S63845 and ABT-199 along with the controls.

FIG. 7A is a graphical representation of the data from a luciferase-based growth assay showing reduced M.tb growth following 6-7 days of treatment with a combination of ABT-199 and S63845, MIK665, AZD5991, or AMG-176 using MDMs from two different donors. FIG. 7B is a graphical representation of the results of a CFU assay showing that combinatorial treatment with ABT-199 and S63845, MIK665, AZD5991, or AMG-176 reduced M.tb growth using MDMs from two different donors.

FIG. 8A is a graphical representation of caspase-3/7 activity over time confirming that the combination of ABT-199 and S63845 induces apoptosis (host cell death) during M.tb infection of human macrophages, a process that controls M.tb growth. FIGS. 8B and 8C are images of western blot analysis showing an increase in caspase-3 and PARP cleavage induced by ABT-199 and S63845 during M.tb infection of human macrophages.

FIG. 9 is a graphical representation of the results of a CFU assay showing that combinatorial treatment with ABT-199 and S63845 significantly reduced growth of multi-drug resistant M.tb in human macrophages.

### DETAILED DESCRIPTION

Reference will now be made to the exemplary embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to one skilled in the relevant art and having possession of this disclosure, are to be considered within the scope of the invention.

As used herein, the singular forms “a”, “an”, and “the” include both singular and plural referents unless the context clearly dictates otherwise. The recitation of numerical ranges by endpoints includes all numbers and fractions subsumed within the respective ranges, as well as the recited endpoints. The term “about” as used herein when referring to a measurable value such as a parameter, an amount, a temporal duration, and the like, is meant to encompass variations of and from the specified value, such as variations of +/−10% or less from the specified value, but can be specifically identified to mean a separate variation, such as +/−5% or less, +/−1% or less, or +/−0.1% or less of and from the specified value. It is to be understood that the value to which the modifier “about” refers is itself also specifically, and preferably, disclosed.

The terms “first,” “second,” “third,” etc. when referring to more than one element or aspect are arbitrary and are to be interpreted based on the context in which they are used. Thus, for example, when referring to two compositions, one having an MCL-1 inhibitor and one having a BCL-2 inhibitor, in some aspects the MCL-1 inhibitor composition is referred to as the “first composition”; in some aspects the BCL-2 inhibitor composition is referred to as the “first composition” depending on the context in which the terms are used.

Apoptosis of infected cells is an important host defense mechanism that prevents the growth of intracellular bacteria and viruses, including M.tb. Induction of macrophage apoptosis leads to reduced M.tb growth and increased survival. Host factors that regulate apoptosis include members of the BCL-2 protein family, which includes (1) pro-survival proteins (MCL-1, BCL-XL, BCL-2, BFL-1/A1, BCL-w, and BCL-b), (2) pro-apoptotic proteins (BAK and BAX), and (3) pro-apoptotic BH3-only motif proteins (NOXA, PUMA, BIM, BID, BIK, BAD, HRK, and BMF). MCL-1 and BCL-2 play critical roles in regulation of apoptosis and, as such, their activity is tightly regulated at the transcriptional, post-transcriptional, and post-translational levels. More particularly, MCL-1 (also known as myeloid leukemia cell differentiation protein MCL-1), is believed to be involved in the down-regulation of apoptosis leading to the maintenance of cell viability. BCL-2 (also known as apoptosis regulator BCL-2) is believed to down-regulate apoptosis cell death in a variety of cell systems by controlling the mitochondrial membrane permeability. BCL-2 is also believed to function in a feedback loop system with caspases.

Provided are methods of treating infectious diseases, including infectious diseases that hijack host cellular machinery to survive. In some aspects, the treatment is a host-directed therapy. In some aspects, the infectious disease is infection with M.tb. Additional infections that can be treated with MCL-1 and BCL-2 inhibitors include those caused by Chlamydia, Neisseria gonorrhoeae and Legionella pneumophila. This combination of MCL-1 and BCL-2 inhibitors can also be used in the treatment of infectious diseases caused by other intracellular pathogens of macrophages like Salmonella, Leishmania, Listeria monocytogenes, Francisella tularensis, Yersinia pestis, Histoplasma, Cocciodomycosis, Blastomycosis and Coxiella burnetii.

Tuberculosis (TB) is a disease caused by the bacterium Mycobacterium tuberculosis, or related mycobacterial species M. bovis, M africanum, M. microti, M. caprae, M pinnipedii, M. canetti, or M. mungi. The disease can be classified into a variety of sub-categories, including pulmonary TB, extrapulmonary TB, miliary TB, and TB meningitis. Within each category, TB can be described as being in an active or latent phase. Without being bound by theory, it is believed that M.tb infection in humans generally occurs when tubercle bacilli dispersed in the air from a subject with active pulmonary TB reach the alveoli of a host, where M.tb is phagocytosed by alveolar macrophages. Bacilli replicate in the macrophages and then diffuse to nearby cells including epithelial and endothelial cells. M.tb can infect a variety of organs such as the brain, larynx, lymph nodes, lungs, spine and kidney. Once the adaptive immune response is elicited, migration to the site of primary infection of neutrophils, lymphocytes and other immune cells form a cellular infiltrate that later assume the typical structure of a granuloma. Fibrotic components cover the granuloma that becomes calcified such that bacilli remain encapsulated inside and protected by the host immune response. Latent M.tb resides in the granuloma and also in cells and tissues not associated with the granuloma (such as in adipocytes). M.tb pathogenesis is discussed further in Torrelles J B, et al. Trends Microbiol. 2017 August; 25(8):688-697. doi: 10.1016/j.tim.2017.03.007, which is incorporated herein by reference in its entirety. M.tb infection is associated with a variety of symptoms, including weakness, fatigue, weight loss, decreased appetite, chills, fever, cough, chest pain, and night sweats. In some cases, subjects infected with M.tb are asymptomatic, such as during latent infection.

TB is classically diagnosed via detection of M.tb from a biological sample of a subject. Such detection can be performed via any available microbiological techniques, such as microscopical analysis, isolation in culture, immunological, or molecular methods. High sensitivity and specificity has been observed in the detection of M.tb in specimens such as sputum, bronchoalveolar lavage or induced sputum for the diagnosis of pulmonary TB. Non-pulmonary forms of TB can be diagnosed using non-pulmonary specimens. Detection of M.tb in the urine or stools can be used to detect systemic infections, and assays capable of detecting mycobacterial components (e.g., lipoarabinomannan, LAM) in the urine have been shown to be helpful to diagnose TB in HIV-infected subjects and immunocompromised patients. Exemplary diagnostic methods of detecting M.tb infection are discussed in Cheon et al., J. Microbiological Methods, 123: 51-61 (2016), which is incorporated herein by reference in its entirety.

Provided are methods of inhibiting the growth of an infectious disease by contacting cells infected with or surrounding a pathogen (e.g., M.tb) with two or more BCL-2 family protein inhibitors. In some aspects, the two or more inhibitors comprise a MCL-1 inhibitor and an inhibitor that targets a separate BCL-2 family protein (e.g., a BCL-2 inhibitor). Some aspects include inhibiting the growth of the infectious disease by contacting macrophages with the inhibitors. Some aspects include inhibiting growth by contacting granulomas with the inhibitors. Some aspects include inducing or enhancing cellular apoptosis in host cells using the inhibitors which control M.tb growth (Arnett E, Schlesinger L S. Immunity. 2021 Aug. 10; 54(8):1625-1627. doi: 10.1016/j.immuni.2021107.010). Some aspects include administering to the subject a therapeutically effective amount of an anti-tuberculosis antibiotic, along with a MCL-1 inhibitor and an inhibitor that targets a separate BCL-2 family protein (e.g., a BCL-2 inhibitor). Examples of anti-tuberculosis antibiotics include first-line anti-tuberculosis drugs, such as isoniazid, rifampin, rifabutin, rifapentine, ethambutol, and pyrazinamide. Other examples of anti-tuberculosis antibiotics include second-line anti-tuberculosis antibiotics, such as cycloserine, capreomycin, kanamycin, amikacin, fluoroquinolones (e.g. levofloxacin and moxifloxacin), ethionamide, protionamide, terizidone, and para-aminosalicylic acid.

Also provided are methods of treating an infectious disease (e.g., M.tb) in a subject by targeting two or more BCL-2 family proteins. Treatment includes any type of measure that imparts a benefit on a patient afflicted with or at risk of developing an infectious disease or symptoms thereof. Such benefits can include slowing, controlling, reversing, or stopping the progression of one or more symptoms of the disease and/or the disease process itself. Such treatment can include, but does not require, a total elimination of all symptoms or a cure of the disease. Treatment can include an inhibition of the growth of a pathogen, killing of the pathogen, or complete elimination of the pathogen.

BCL-2 family proteins can be targeted by administering BCL-2 family protein inhibitors to the subject. Some aspects include administering two or more BCL-2 family protein inhibitors to the subject. Exemplary BCL-2 family protein inhibitors are set forth in Ashkenazi et. al, Nature Reviews Drug Discovery, 16(4): 273-284 (2017), which is incorporated by reference in its entirety. In some aspects, the two or more BCL-2 family protein inhibitors are specific inhibitors. In some aspects, the specific inhibitors have reduced toxicity as compared to a pan inhibitor. In some aspects, one or more (or all) of the administered inhibitors are not pan inhibitors.

In an embodiment, the BCL-2 family protein inhibitors (e.g., a MCL-1 inhibitor and a BCL-2 inhibitor) are administered to treat a subject having TB. In an embodiment, the BCL-2 family protein inhibitors (e.g., a MCL-1 inhibitor and a BCL-2 inhibitor) are administered to treat a subject having TB in addition to other co-morbidities, such as other infectious or metabolic diseases. In an embodiment, the infectious disease is caused by HIV. Some aspects include administering the inhibitors to a subject having active TB. Some aspects include administering the inhibitors to a subject having latent TB. Some aspects include administering the inhibitors to a subject having multi-drug resistant TB. Some aspects include administering the inhibitors to a subject having one or more symptoms of TB. Some aspects include administering the inhibitors to an asymptomatic subject. In some aspects, the subject has been identified as having an infectious disease or being at risk of having an infectious disease. In some aspects, the subject has been diagnosed as having the infectious disease in accordance with methods described herein. The subject can be any mammalian subject, such as a human subject.

Some aspects include administering a MCL-1 inhibitor to a subject. Such inhibitors can target MCL-1 nucleic acids or proteins. In some aspects, the MCL-1 inhibitor is a BH3 mimetic. Exemplary MCL-1 inhibitors include S63845, A-1210477, MIM-1, MIK665/S-64315, 483-LM, AZD5991, AMG-176, AMG-397, UMI-77, VU661013, and JKY-5-037. Some aspects include administering combinations of MCL-1 inhibitors to the subject, such as any combination or two or more of the foregoing MCL-1 inhibitors. Exemplary MCL-1 inhibitors are set forth in Hird et. al, Pharmacology & Therapeutics, 198: 59-67 (2019), which is incorporated by reference in its entirety.

Some aspects include administering a BCL-2 inhibitor to a subject. Such inhibitors can target BCL-2 nucleic acids or proteins. In some aspects, the BCL-2 inhibitor is a BH3 mimetic. Exemplary BCL-2 inhibitors include venetoclax (ABT-199), ABT-263, S55746, and ABT-737. Some aspects include administering combinations of BCL-2 inhibitors to the subject, such as any combination or two or more of the foregoing BCL-2 inhibitors. An embodiment includes administering to a subject in need thereof a combination of venetoclax as a BCL-2 inhibitor and S63845 as the MCL-1 inhibitor. Another embodiment includes administering to a subject in need thereof a combination of venetoclax as a BCL-2 inhibitor and MIK665/S-64315 as the MCL-1 inhibitor. Another embodiment includes administering to a subject in need thereof a combination of venetoclax as a BCL-2 inhibitor and AZD5991 as the MCL-1 inhibitor.

Some aspects include administering two or more BCL-2 family protein inhibitors (e.g., a MCL-1 inhibitor and a BCL-2 inhibitor) in the same composition. Some aspects include administering two or more BCL-2 family protein inhibitors (e.g., a MCL-1 inhibitor and a BCL-2 inhibitor) in separate compositions.

As used herein, unless otherwise noted, the terms “treating”, “treatment” and the like, shall include the management and care of a subject or patient (preferably mammal, more preferably human) for the purpose of combating an infection and includes the administration of a compound of the present invention to prevent the onset of the symptoms or complications, alleviate the symptoms or complications, or eliminate the infection. The term “subject” as used herein, refers to an animal, preferably a mammal, most preferably a human, who has been the object of treatment, observation or experiment. Preferably, the subject has experienced and/or exhibited at least one symptom of the infection to be treated and/or prevented. Some aspects include administering to a subject in need thereof a therapeutically effective amount of the inhibitors. As used herein, the phrase “therapeutically effective amount” means a dosage that provides the specific pharmacological response for which the inhibitor is administered in a subject in need of such treatment. It is emphasized that a therapeutically effective amount or therapeutic level of an inhibitor will not always be effective in treating the disease, even though such dosage is deemed to be a therapeutically effective amount by those of skill in the art. For convenience only, exemplary dosages, drug delivery amounts, therapeutically effective amounts and therapeutic levels are provided herein with reference to adult human subjects. Those skilled in the art can adjust such amounts in accordance with standard practices as needed to treat a specific subject and/or condition/disease.

“Inhibitors” as described herein also include pharmaceutically acceptable, pharmacologically active derivatives of the inhibitors specifically mentioned herein, including but not limited to salts, esters, amides, prodrugs, metabolites, analogs and the like. The inhibitors can be administered in a pharmaceutical composition containing one or more pharmaceutically acceptable excipients or carriers, and optionally other therapeutic and/or prophylactic ingredients. Such excipients include liquids such as water, saline, glycerol, polyethylene glycol, ethanol, and the like. Suitable excipients for non-liquid formulations are also known to those of skill in the art. A discussion of pharmaceutically acceptable excipients and salts is available in Remington's Pharmaceutical Sciences, 18th Edition (Easton, Pennsylvania: Mack Publishing Company, 1990).

“Pharmaceutically acceptable carrier” means a diluent, adjuvant, excipient or carrier with which an inhibitor is administered. Such carriers are well known in the pharmaceutical art, and are described, for example, in Remington's Pharmaceutical Sciences, 18th Edition (Easton, Pennsylvania: Mack Publishing Company, 1990). For example, sterile saline and phosphate-buffered saline at physiological pH can be used. Such carriers can be liquids. In some aspects, the carriers include saline, gum acacia, gelatin, starch paste, talc, keratin, colloidal silica, urea, or other similar compounds. Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used. Additionally, auxiliary substances, such as wetting or emulsifying agents, biological buffering substances, surfactants, and the like, can be present in such carriers. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, aerosols, sprays, suspensions, emulsions, sustained-release formulations, creams, ointments, lotions or the like, preferably in unit dosage form suitable for single administration of a precise dosage. The compositions can include an effective amount of the selected inhibitor in combination with a pharmaceutically acceptable carrier and, in addition, can include other pharmaceutical agents, adjuvants, diluents, buffers, and the like. The inhibitors and compositions may be administered by any other convenient route. For example, methods of administration include but are not limited to intradermal, intramuscular, intraperitoneal, intravenous, subcutaneous, intranasal, epidural, oral, sublingual, intranasal, intracerebral, intravaginal, transdermal, rectal, by inhalation, or topically. Administration can be systemic or local.

Suitable dosage ranges for oral administration are 0.01 milligrams to 5000 milligrams per kilogram of body weight, such as 0.1 milligrams to 3000 milligrams per kilogram of body weight, or 1 to 100 milligrams per kilogram of body weight. Suitable dosage ranges for intravenous (i.v.) administration are 0.01 milligrams to 1000 milligrams per kilogram body weight, 0.1 milligram to 350 milligrams per kilogram body weight, and 1 milligram to 100 milligrams per kilogram body weight. Suitable dosage ranges for intranasal administration are generally about 0.01 pg/kg body weight to 1 mg/kg body weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the compounds of the invention for topical administration are in the range of 0.001 milligram to 1 milligram, depending on the area to which the compound is administered. Effective doses may be extrapolated from dose-response curves derived from in vitro or animal model test systems. The dose should be adjusted to suit the individual to whom the composition is administered and will vary with age, weight and metabolism of the individual. The exact amount of the composition required will vary from subject to subject, depending on the species, age, weight and general condition of the subject, the particular inhibitor used, its mode of administration and the like.

Some aspects include administering the inhibitor or composition one or more times per day. Some aspects include administering the inhibitors or compositions daily, every other day, or at some other interval. Some aspects include administering the inhibitors or compositions for a period of at least 1 week, at least 2 weeks, at least 3 weeks, at least 4 weeks, at least 6 weeks, at least 2 months, at least 3 months, at least 4 months, at least 5 months, or at least 6 months.

When two or more inhibitors are administered in separate compositions, the characteristics of the compositions, routes of administration, dosages, and dosing regimens are independently determined for each composition. Thus, the compositions may have different dosage forms, routes of administration, dosages, dosing regimens, etc.

In an embodiment, the MCL-1 inhibitors are prepared for oral (i.e. a tablet, capsule, suspension or liquid), injection, intravenous, intramuscular, subcutaneous, intraperitoneal, or parenteral administration. Embodiments include administering a composition containing a MCL-1 inhibitor to a subject at a dosage of about 2 mg/kg to about 50 mg/kg. In some aspects, the dosage is about 2 mg/kg, about 5 mg/kg, about 7.5 mg/kg, about 10 mg/kg, about 15 mg/kg, about 20 mg/kg, about 25 mg/kg, about 30 mg/kg, about 40 mg/kg, or about 50 mg/kg. In some aspects, the dosage is 2 mg/kg, 5 mg/kg, 7.5 mg/kg, 10 mg/kg, 15 mg/kg, 20 mg/kg, 25 mg/kg, 30 mg/kg, 40 mg/kg, or 50 mg/kg. In some aspects, the composition is administered to the subject daily or twice per week. In some aspects, the composition is administered to the subject at least 3 days per week. In some aspects, the composition is administered to the subject 3 days per week. In some aspects, the composition is administered to the subject 3 days per week or more for at least 1 month. Some aspects include further administering a composition containing a BCL-2 inhibitor, along with an anti-TB treatment regimen.

In an embodiment, the BCL-2 inhibitors are prepared for oral (i.e. a tablet, capsule, suspension or liquid), injection, intravenous, intramuscular, subcutaneous, intraperitoneal, or parenteral administration. Some aspects include administration of a composition containing a BCL-2 inhibitor to a subject at a dosage of from about 2 mg/kg to about 300 mg/kg. In some aspects, the dosage is about 2 mg/kg, about 5 mg/kg, about 7.5 mg/kg, about 10 mg/kg, about 15 mg/kg, about 20 mg/kg, about 25 mg/kg, about 30 mg/kg, about 40 mg/kg, about 50 mg/kg, about 75 mg/kg, about 100 mg/kg, about 150 mg/kg, about 200 mg/kg, about 250 mg/kg, or about 300 mg/kg. In some aspects, the dosage is 2 mg/kg, 5 mg/kg, 7.5 mg/kg, 10 mg/kg, 15 mg/kg, 20 mg/kg, 25 mg/kg, 30 mg/kg, 40 mg/kg, 50 mg/kg, 75 mg/kg, 100 mg/kg, 150 mg/kg, 200 mg/kg, 250 mg/kg, or 300 mg/kg. In some aspects, the composition is administered to the subject daily or twice per week. In some aspects, the composition is administered to the subject at least 5 days per week. In some aspects, the composition is administered to the subject 5 days per week. In some aspects, the composition is administered to the subject 5 days per week or more for at least 1 month. In some embodiments, patients are administered a combination of a BCL-2 inhibitor and a MCL-1 inhibitor. In some embodiments, patients are administered a combination of a BCL-2 inhibitor and a MCL-1 inhibitor, along with an anti-TB treatment regimen.

The following examples are included as illustrative of the compositions and methods described herein. The examples are in no way intended to limit the scope of the invention. Other aspects will be apparent to those skilled in the art. For example, in each instance herein any of the terms “comprising”, “consisting essentially of” and “consisting of” may be replaced with either of the other two terms; moreover, any of the terms may be used in reference to features disclosed herein.

### EXAMPLES

### Example 1: MCL-1 and BCL-2 Contribute to M.tb Growth in Marine Macrophages

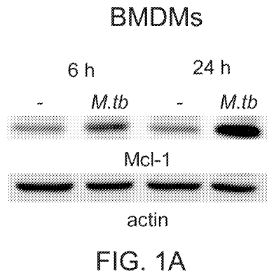

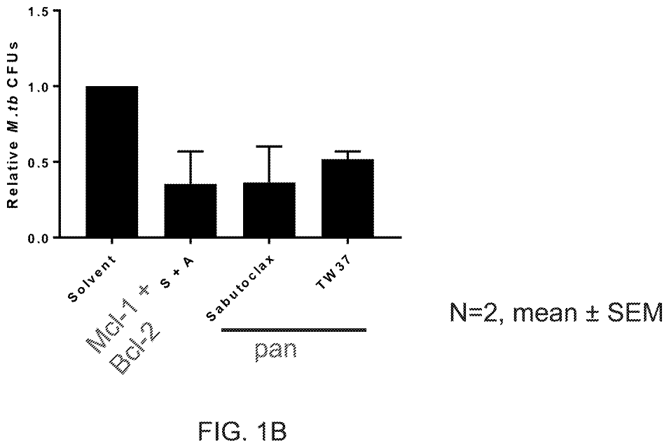

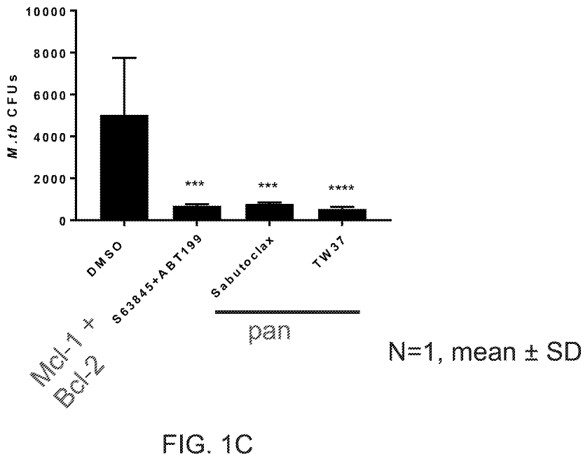

Murine BMDMs were obtained from tibias and femurs of C57BL/6 mice, then cultured in 30% L cell conditioned media, 20% heat-inactivated fetal bovine serum (HI-FBS), 50.3 μM beta-mercaptoethanol, 10 units Penicillin/Streptomycin in DMEM for about 6 days. BMDMs were frozen in media with 10% dimethylsulfoxide. The day before the experiment BMDMs were thawed and plated in DMEM supplemented with 10 units Penicillin/Streptomycin and 10% HI-FBS (as published in Arnett et al. Cell Microbiol. (2011)). BMDMs were infected with single cell suspension of M.tb at MOI 1 (multiplicity of infection of 1) for 2 hours. BMDMs were then washed and incubated in 2% HI-FBS/DMEM for 6 or 24 hours. Cells were washed with PBS, then lysed with TN1 lysis buffer (125 mM NaCl, 50 mM Tris, 10 mM EDTA, 1% Triton X-100, 10 mM Na₄PO₇, 10 mM NaF with 10 mM Na₃VO₄, 10 μg/ml aprotinin, and 10 μg/ml leupeptin) at 4° C. Lysates were centrifuged (10,000 g, 4° C., 10 min) to remove cell debris, then a Pierce BCA assay was performed to determine protein concentration. Equivalent amounts of denatured and reduced protein were separated by SDS-PAGE and analyzed by Western blot using antibodies against Mcl-1 (Santa-Cruz, Dallas, TX), and β-actin (Santa-Cruz). Western blot shows that BMDMs infected with M.tb exhibit increased expression of MCL-1 at 6 hours and 24 hours after infection, as shown in FIG. 1A (in the figure, actin expression is used as a control). These results show that M.tb drives expression of MCL-1 in murine macrophages in alignment with previously reported data in primary human macrophages.

Murine BMDMs were isolated as described above. For lung macrophage isolation, BALB/c mice were euthanized by CO₂ asphyxiation. Following perfusion through the heart with 10 ml of PBS containing 50 U/ml of heparin, the lungs were removed and placed in DMEM supplemented with 10% HI-FBS, 10 mM HEPES buffer, 2× minimal non-essential amino acids, 2 mM L-glutamine and 1 U/ml Penicillin/Streptomycin (cDMEM). The lung lobes were dissociated into single cell suspensions by enzymatic degradation with collagenase XI and type IV bovine pancreatic DNase for 30 min at 37° C., 5% CO₂ and 95% relative humidity followed by a mechanical dissociation process (gentleMACS dissociator, Miltenyi Biotec Inc.). cDMEM was added to dilute enzymatic activity and the lung pieces were pressed through sterile 70 m nylon mesh screens to obtain single cell suspensions. Residual red blood cells were lysed using lysis buffer (0.15 M NH₄Cl, 1 mM KHCO₃) for 3 min at room temperature followed by washing with cDMEM. Cells were centrifuged at 200 g for 7 min at 4° C. and re-suspended in cDMEM then allowed to attach to tissue culture treated dishes for 2 hours prior to washing. BMDM and lung macrophages were washed with DMEM then infected with M.tb at MOI 1 for 2 hours, washed, then treated with a solvent (DMSO control), 30 μM pan inhibitor sabutoclax, 30 μM pan inhibitor TW37, or a combination of 10 μM S63845 and 10 μM ABT-199 in 2% HI-FBS/DMEM for 2-4 days. Intracellular growth of M.tb was evaluated using CFU assays, in which macrophages were lysed, and lysates were diluted and plated on 7H11 agar. The number of CFUs was enumerated after growth for 3-4 weeks at 37° C.

FIGS. 1B and 1C are graphical representations of the results of a CFU (colony forming unit) assay showing that treating murine BMDM or lung macrophages infected with M.tb with a combination of S63845 and ABT-199 resulted in a reduction in the number of M.tb CFUs comparable to that achieved with pan BCL-2 family inhibitors sabutoclax and TW37. FIG. 1B sets forth CFUs normalized based on the solvent/DMSO control in BMDMs, and FIG. 1C sets forth the absolute number of CFUs in lung macrophages. As shown in FIGS. 1B and 1C, treatment with the combination of S63845 and ABT-199 resulted in a reduction in the number of M.tb CFUs comparable to that achieved with pan inhibitors sabutoclax and TW37 (FIG. 1B sets forth mean CFUs normalized based on the control in BMDMs±standard error of the mean for 2 separate experiments after 4 days of treatment; FIG. 1C sets forth mean number of CFUs±standard deviation in lung macrophages after 2 days of treatment, ***p<0.001, ****p<0.0001 indicates results are significantly different from DMSO solvent control). The results show that the combinatorial therapy reduces M.tb growth in murine BMDMs and lung macrophages.

### Example 2: MCL-1 and BCL-2 Combinatorial Therapy Limits M.tb Growth in Human Granulomas

In vitro TB granulomas were generated as described in Arnett et al., PLOS Pathogens, 14(6): e1007100 (2018). Briefly, heparinized blood was collected from healthy Mantoux tuberculin skin test (TST) and/or IFNγ release assay (IGRA)-positive individuals, following approved IRB protocols. Blood was layered on a Ficoll-Paque cushion to allow for collection of peripheral blood mononuclear cells (PBMCs). PBMCs were then were infected with M.tb at MOI 1 in RPMI with 10% autologous serum, then incubated at 37° C./5% C02. After 1 day, inhibitors were added, and additional serum was added after 4 days. Cells were incubated for a total of 4 or 7 days before cells were lysed and M.tb intracellular growth was enumerated with CFUs. The inhibitors and concentrations tested are set forth in Table 1.

TABLE 1
MCL-1 and BCL-1 inhibitors and concentrations

| Experiment | | | |
|---|---|---|---|
| condition. | Inhibitor | Concentration | |
| 1 | DMSO (negative control) | — | |
| 2 | Sabutoclax (positive control) | 30 | μM |
| 3 | ABT-199 (BCL-1 inhibitor) | 1 | μM |
| 4 | ABT-199 (BCL-1 inhibitor) | 10 | μM |
| 5 | A-1210477 (MCL-1 inhibitor) | 30 | μM |
| 6 | MIM-1 (MCL-1 inhibitor) | 30 | μM |
| 7 | S63845 (MCL-1 inhibitor) | 1 | μM |
| 8 | S63845 (MCL-1 inhibitor) | 10 | μM |
| 9 | ABT-199 (BCL-1 inhibitor) + | 1 | μM |
| | A-1210477 (MCL-1 inhibitor) | 30 | μM |
| 10 | ABT-199 (BCL-1 inhibitor) + | 1 | μM |
| | MIM-1 (MCL-1 inhibitor) | 30 | μM |
| 11 | ABT-199 (BCL-1 inhibitor) + | 1 | μM |
| | S63845 (MCL-1 inhibitor) | 1 | μM |
| 12 | ABT-199 (BCL-1 inhibitor) + | 1 | μM |
| | S63845 (MCL-1 inhibitor) | 10 | μM |
| 13 | ABT-199 (BCL-1 inhibitor) + | 10 | μM |
| | A-1210477 (MCL-1 inhibitor) | 30 | μM |
| 14 | ABT-199 (BCL-1 inhibitor) + | 10 | μM |
| | MIM-1 (MCL-1 inhibitor) | 30 | μM |
| 15 | ABT-199 (BCL-1 inhibitor) + | 10 | μM |
| | S63845 (MCL-1 inhibitor) | 1 | μM |
| 16 | ABT-199 (BCL-1 inhibitor) + | 10 | μM |
| | S63845 (MCL-1 inhibitor) | 10 | μM |

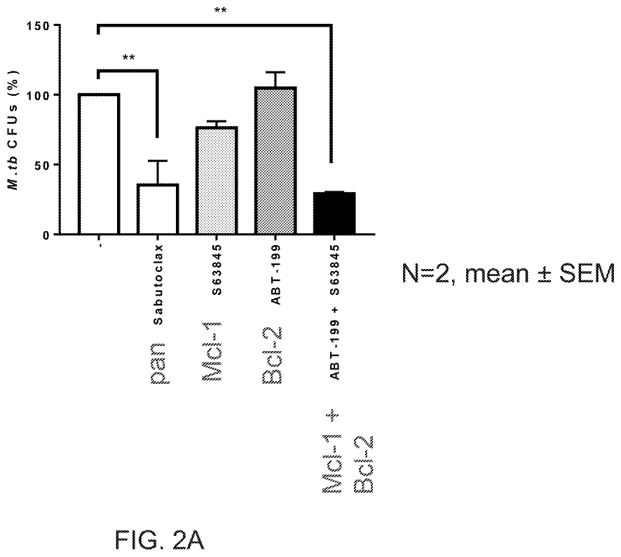

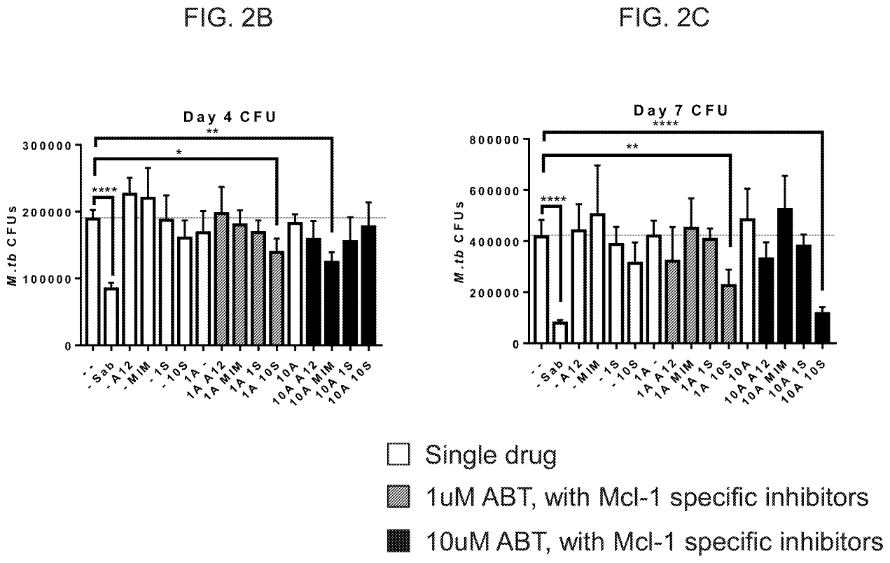

As shown in FIGS. 2A-2C, combination therapy with 10 μM ABT-199 and 10 μM S63845 show reduced M.tb growth after 7 days as compared to treatment with MCL-1 or BCL-2 inhibitors alone. FIG. 2A is a graphical representation of the results of a CFU assay showing that treating human granulomas infected with M.tb with a combination of 10 μM S63 845 and 10 μM ABT-199 resulted in reduced M.tb growth as compared to treatment with MCL-1 or BCL-2 inhibitors alone. FIGS. 2B and 2C are graphical representations of the results of a CFU assay showing that treating human granulomas infected with M.tb with a combination of 10 μM ABT-199 and 10 μM S63845 reduced M.tb growth in granulomas to a similar extent as the general BCL-2 family inhibitor sabutoclax at 7 days (FIG. 2C) as compared to treatment with MCL-1 or BCL-2 inhibitors alone, or combinatorial treatment with BCL-2 inhibitor ABT-199 and select other MCL-1 inhibitors with reduced activity relative to the MCL-1 inhibitor S63845. Results shown in FIG. 2A are mean±standard error of the mean for 2 experiments using PBMCs from 2 different individuals, **p<0.01 indicates a significant reduction in M.tb growth with combinatorial or sabutoclax treatment, relative to untreated control. Results shown in FIGS. 2B and 2C are representative of 2 experiments and show that combination therapy with 10 μM ABT-199 and 10 μM S63845 reduced M.tb growth in granulomas to a similar extent as the general BCL-2 family inhibitor sabutoclax at 7 days as compared to treatment with MCL-1 or BCL-2 inhibitors alone (**** p<0.0001). Combination therapy with 1 μM ABT-199 and 10 μM S63845 also reduced M.tb growth in granulomas (**p<0.01). Thus, the results show that inhibiting multiple BCL-2 family proteins reduces M.tb growth in human in vitro granulomas.

### Example 3: MCL-1 and BCL-2 Combinatorial Therapy Limits M.tb Growth in Human Macrophages

Human monocyte-derived macrophages (MDMs) were generated as previously described in Arnett et al., PLOS Pathogens, 14(6): e1007100 (2018). Briefly, heparinized blood was layered on a Ficoll-Paque cushion to allow for collection of PBMCs. PBMCs were then cultured in RPMI with 20% autologous serum in Teflon wells at 37° C./5% CO₂. After 5 days, PBMCs containing MDMs were harvested and adhered to tissue culture dishes in RPMI with 10% autologous serum. After 2-3 hours, lymphocytes were washed away and MDMs were incubated in RPMI with 10% autologous serum. Such MDM monolayers are 99% pure and viable. The following day, MDMs from 2 different donors were infected with a M.tb reporter strain expressing the bacterial Lux operon (M.tb-lux) at MOI 1 for 2 hours. The MDMs were washed and incubated with 2% serum +/− inhibitors for the remaining time. The MDMs were treated with inhibitors at the concentrations set forth in Table 2. Mtb-lux was assessed daily (up to 4 days for one donor; up to 7 days for the other).

TABLE 2
Inhibitors and concentrations used to treat MDMs

| Experiment No. | Inhibitor | Concentration | |
|---|---|---|---|
| 1 | DMSO (negative control) | — | |
| 2 | Sabutoclax (positive control) | 30 | μM |
| 3 | ABT-199 | 0.1 | μM |
| 4 | ABT-199 | 1 | μM |
| 5 | ABT-199 | 10 | μM |
| 6 | S63845 | 0.1 | μM |
| 7 | S63845 | 1 | μM |
| 8 | S63845 | 10 | μM |
| 9 | ABT-199 | 10 | μM |
| | S63845 | 10 | μM |

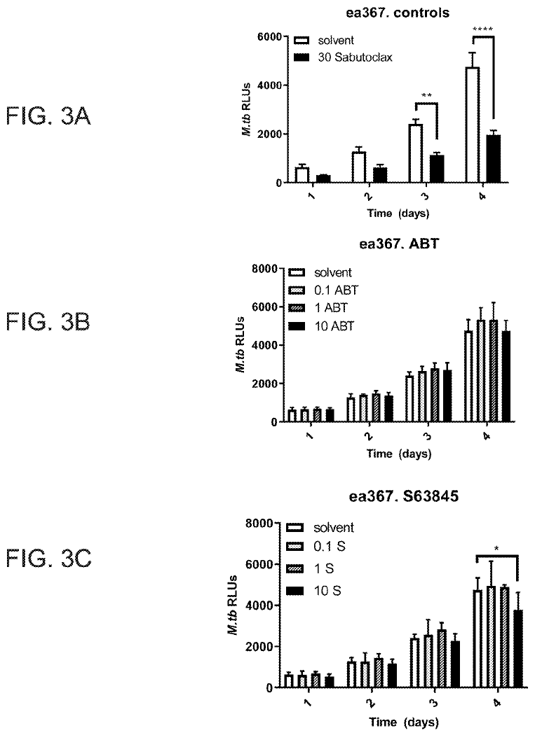

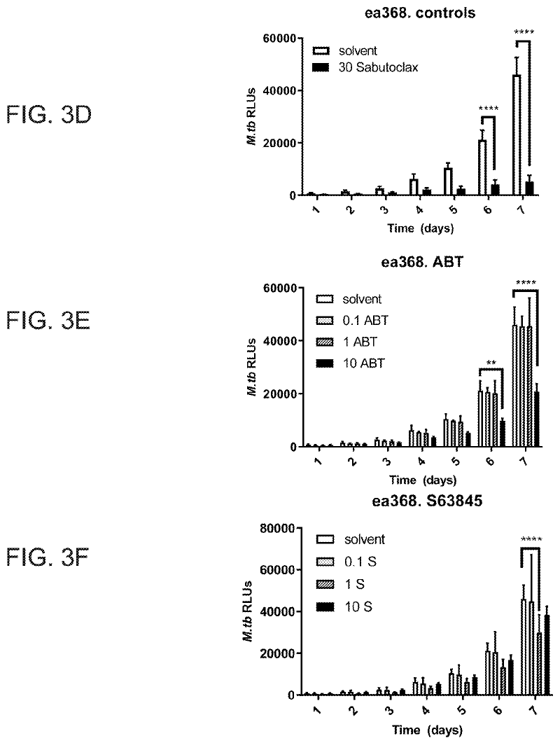

FIGS. 3A-3C show that treatment with 10 μM S63845 reduced M.tb growth in donor 1 after 4 days, but to a limited extent relative to the pan inhibitor Sabutoclax. FIGS. 3A-3C are graphical representations of the data from a luciferase-based growth assay showing human monocyte-derived macrophages (MDMs) treated with the general BCL-2 family inhibitor sabutoclax (FIG. 3A), ABT-199 (FIG. 3B), and S63845 (FIG. 3C). Treating human monocyte-derived macrophages (MDMs) with 10 μM S63845 (FIG. 3C) reduced M.tb growth after 4 days, but to a lesser extent than the general BCL-2 family inhibitor sabutoclax (FIG. 3A). Results are mean±standard deviation, *p<0.05, **p<0.01, ***p<0.001.

FIGS. 3D-3F show reduced M.tb growth in donor 2 after (i) 7 days of treatment with 1 μM S63845 and (ii) 6 days of treatment with 10 μM ABT-199, but to a limited extent relative to the pan inhibitor Sabutoclax. FIGS. 3D-3F are graphical representations of the data from a luciferase-based growth assay using MDMs from a different donor showing MDMs treated with the general BCL-2 family inhibitor sabutoclax (FIG. 3D), ABT-199 (FIG. 3E), and S63845 (FIG. 3F). There was reduced growth of M.tb in MDMs after (i) 7 days of treatment with 1 μM S63845 (FIG. 3F) and (ii) 6 days of treatment with 10 μM ABT-199 (FIG. 3E). Results are mean standard deviation, **p<0.01, ****p<0.0001.

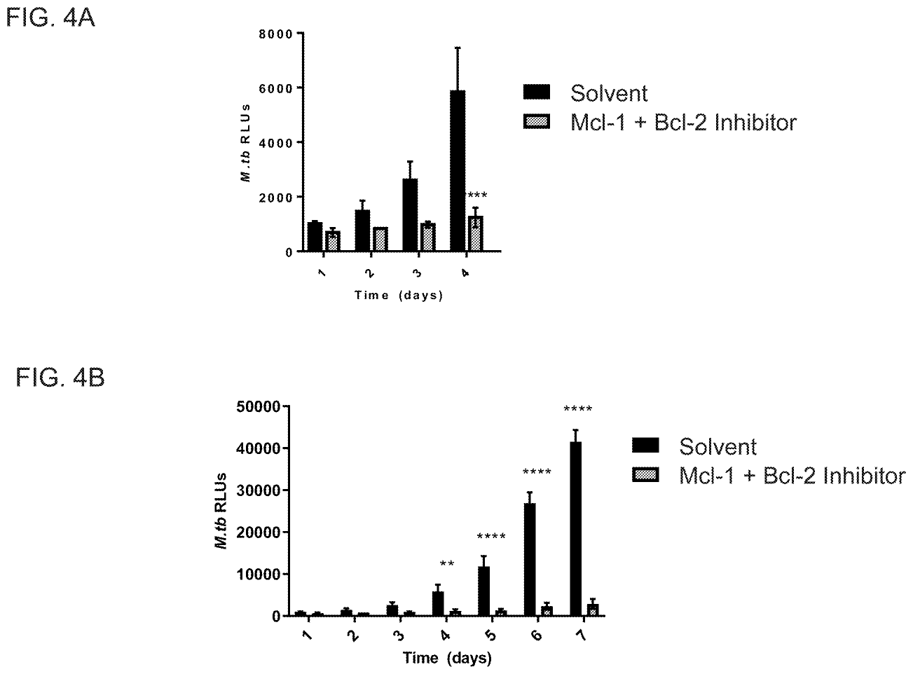

In contrast, FIGS. 4A and 4B are graphical representations of the data from a luciferase-based growth assay showing reduced M.tb growth following 4 days of treatment with a combination of ABT-199 and S63845 using MDMs from two different donors. Results are mean±standard deviation, **p<0.01, ***p<0.001, ****p<0.0001.

Thus, inhibiting multiple BCL-2 family proteins reduces M.tb growth in human macrophages more effectively then inhibiting only one BCL-2 protein.

### Example 4: HIV is Found in M.tb/HIV Co-Infected Granulomas

In vitro TB granulomas were generated as described above, with the following modifications. Blood was collected from TST and/or IGRA-negative individuals, following approved IRB protocols. Blood was layered on a Ficoll-Paque cushion to allow for collection of PBMCs, then CD4 T cells were isolated by negative selection. Purified CD4 T cells were stimulated with anti-CD3/CD28 antibodies. After 3 days, washed CD4 T cells were infected with HIV at MOI 0.01. After 72 hours, cells were washed then mixed with freshly isolated PBMCs from the same TST/IGRA-negative individual and infected with M.tb at MOI 1 in RPMI with 10% autologous serum. Cells were incubated for a total of 5 days then cells were washed, fixed, and HIV RNA detected with RNAscope (green) and cell nuclei with DAPI (blue) using a Zeiss LSM 800 fluorescence microscope.

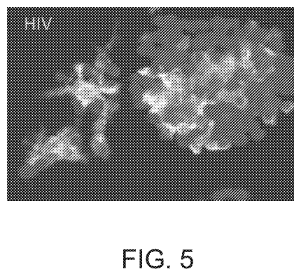

FIG. 5 is a microscope image showing HIV present in a M.tb/HIV co-infected in vitro granuloma. HIV is stained in green, and the human cells in the granuloma are stained in blue. Based on the data presented here, Mcl-1 and Bcl-2 inhibitors can reduce the infectious load in co-infected granulomas, similar to M.tb infected granulomas.

### Example 5: MCL-1 and BCL-2 Combinatorial Therapy Limits M.tb Growth in an Animal Model

BALB/c mice were treated with ABT-199 by oral gavage 5 days/week+S63845 3 days/week intravenously for 4 weeks in accordance with the following groups: (i) control, i.e., no inhibitor administered, (ii) 25 mg/kg ABT-199+3.75 mg/kg S63845, (iii) 50 mg/kg ABT-199+7.5 mg/kg S63845, and (iv) 100 mg/kg ABT+15 mg/kg S63845. Toxicology studies are conducted to evaluate safety of administered doses. Histology analysis confirmed no inflammation in the lungs, liver, kidney, or spleen of animals from any of the groups, confirming the evaluated doses are safe.

Separately, BALB/c mice were infected with M.tb using a low dose aerosol (100 CFU). 4 weeks after infection, mice were treated in accordance with the following groups: (i) negative control, i.e., no inhibitor administered, placebo, (ii) 100 mg/kg ABT+15 mg/kg S63845, (iii) 150 mg/kg ABT+25 mg/kg S63845 and (iv) positive control, i.e., administration of a M.tb antibiotic such as isoniazid. Efficacy of administered doses is evaluated.

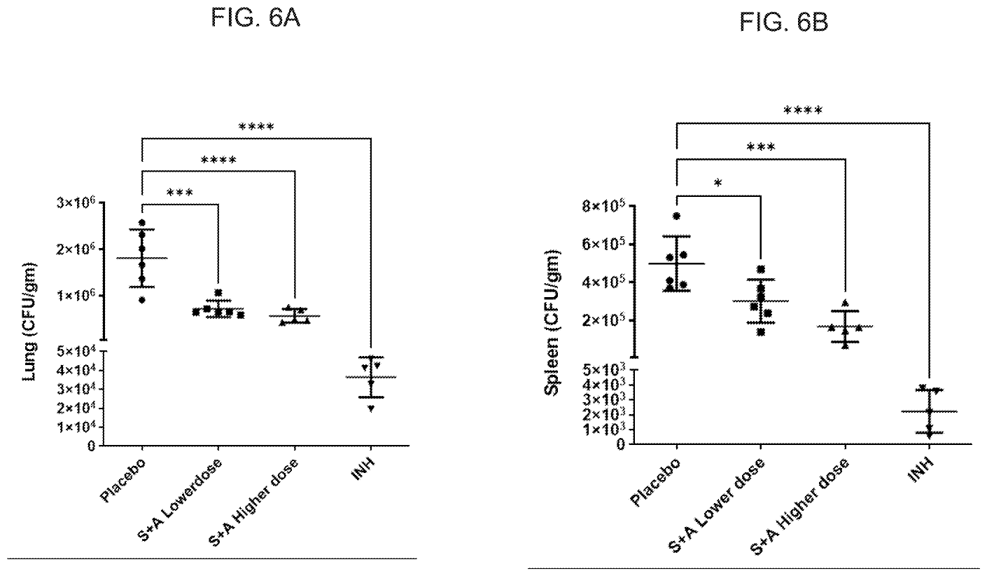

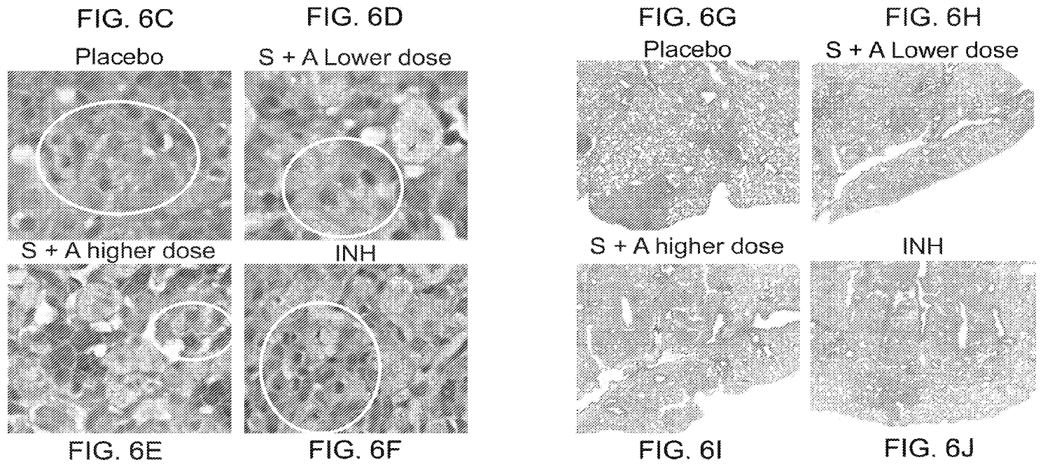

FIGS. 6A-6J show that treatment with 100 mg/kg ABT+15 mg/kg S63845 (S+A Lower dose), 150 mg/kg ABT+25 mg/kg S63845 (S+A Higher dose), or the positive control isoniazid (INH) significantly reduced M.tb growth in murine lungs (FIG. 6A, FIGS. 6D-6F, and FIGS. 6H-6J) and spleen (FIG. 6B), relative to the placebo control (FIGS. 6A-6B, 6C, and 6G). FIGS. 6C-6F shows acid fast stained M.tb in the mouse lung, with a reduction in bacteria for mice treated with 100 mg/kg ABT+15 mg/kg S63845 (S+A Lower dose), 150 mg/kg ABT+25 mg/kg S63845 (S+A Higher dose), or the positive control isoniazid (INH) relative to the placebo control, the white circles indicate the areas in the image with the red M.tb. FIGS. 6H-6J shows hematoxylin and eosin stain of murine lungs, and indicates reduced inflammation in the mice treated with 100 mg/kg ABT+15 mg/kg S63845 (S+A Lower dose), 150 mg/kg ABT+25 mg/kg S63845 (S+A Higher dose), or the positive control isoniazid (INH) relative to the placebo control (FIG. 6G). Results are mean±standard deviation of n=1, with 5-6 mice per group, *p<0.05, ***p<0.001, ****p<0.0001. Thus, inhibiting multiple BCL-2 family proteins reduces M.tb growth and inflammation in animal models.

### Example 6: Combinatorial Therapy with Additional Combinations of Mcl-1 and Bcl-2 Inhibitors Reduces M.tb Growth in Human Macrophages

MDMs from 2 different donors were infected with a M.tb reporter strain expressing the bacterial Lux operon (M.tb-lux) at MOI of 1 for 2 hours. The MDMs were washed and incubated with 2% serum +/−1 μM or 10 μM inhibitors for the remaining time. M.tb-lux was assessed daily and CFU after 4 days.

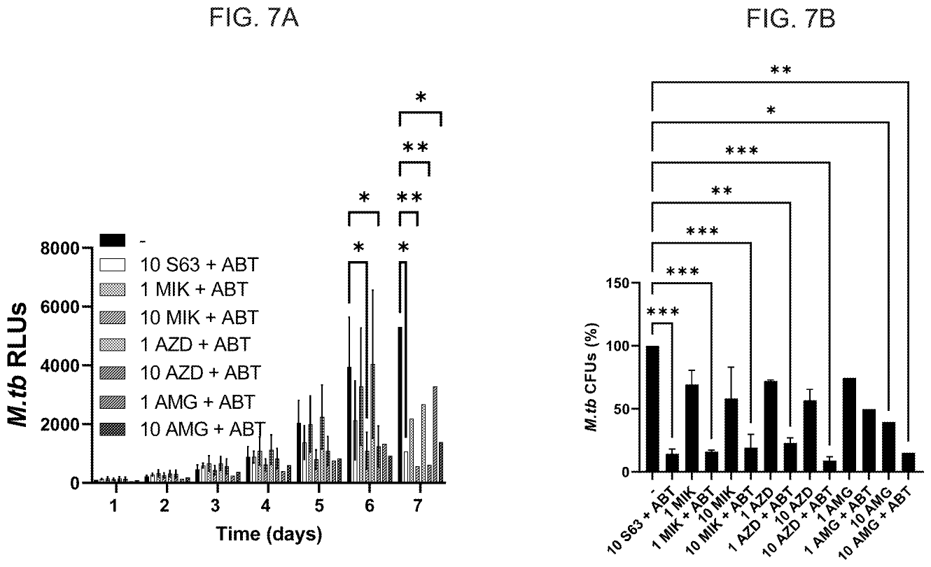

FIGS. 7A and 7B demonstrate that combinatorial treatment with additional Mcl-1 and Bcl-2 inhibitors reduced M.tb growth in human macrophages. FIG. 7A is a graphical representation of the data from a luciferase-based growth assay showing human MDMs treated with 10 μM of the BCL-2 inhibitor ABT-199 and 1 or 10 μM of the Mcl-1 inhibitors S63845, MIK665, AZD5991, or AMG-176. Treating human MDMs with 10 μM ABT-199+10 μM S63845, MIK665, AZD5991, or AMG-176 significantly reduced M.tb growth after 6 days, and the combination of ABT-199 with MIK665 or AZD5991 was the most potent. FIG. 7B is a graphical representation of CFU data from human MDMs treated with 10 μM of the BCL-2 inhibitor ABT-199+/−1 or 10 μM of the Mcl-1 inhibitors S63845, MIK665, AZD5991, or AMG-176. Similar to FIG. 7A, treating human MDMs with 10 μM ABT-199+10 μM S63845, MIK665, AZD5991, or AMG-176 significantly reduced M.tb growth, and this readout could detect a significant reduction in growth after just 4 days, with the combinatorial therapy being more potent than either inhibitor alone. Results are mean±standard error of the mean, n=1-2, *p<0.05, **p<0.01, ***p<0.001. These results show that clinical stage Mcl-1 and Bcl-2 inhibitors reduce M.tb growth in macrophages.

### Example 7: Combinatorial Therapy with Mcl-1 and Bcl-2 Inhibitors Induces Apoptosis in M.tb Infected Human Macrophages

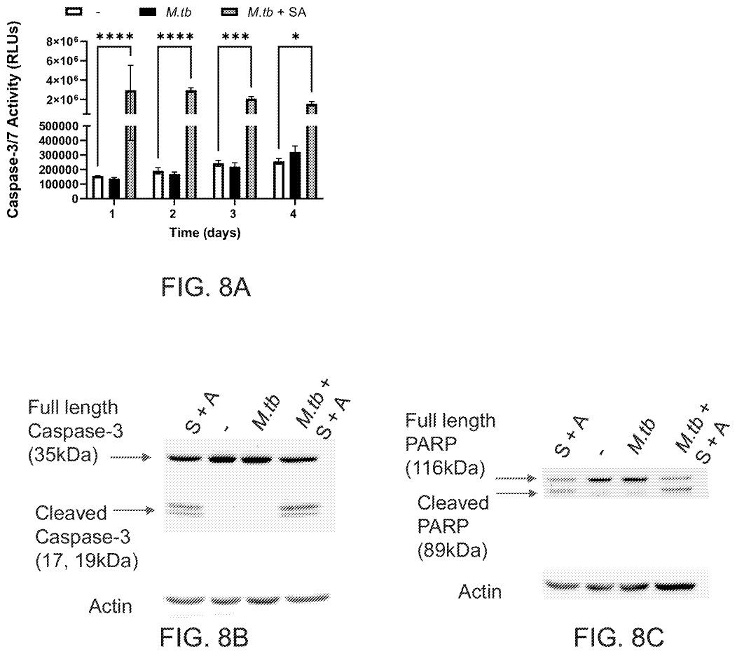

MDMs were infected with M.tb at MOI 1 for 2 hours. The MDMs were washed and incubated with 2% serum +/−10 μM ABT-199 and 10 μM S63845. As a read-out of apoptosis, caspase-3/7 activity was measured with the Caspase-Glo 3/7 Assay System (Promega) daily for 4 days (FIG. 8A). In addition, cells were lysed after 4 hours with TN1 lysis buffer, and equivalent amounts of denatured and reduced protein were separated by SDS-PAGE and analyzed by Western blot using antibodies against Caspase-3, PARP (Cell Signaling), and 3-actin (Santa-Cruz). Cleavage of caspase-3, and caspase-3/7 activity and PARP cleavage leads to apoptosis.

FIG. 8A is a graphical representation of caspase-3/7 activity and shows that 10 μM ABT-199 and 10 μM S63845 induces caspase-3/7 activity during M.tb infection of human macrophages. Results are mean±standard deviation from 1 experiment, *p<0.05, ***p<0.001, ****p<0.0001 indicates a significant increase in apoptosis in M.tb infected macrophages treated with ABT-199 and 10 μM S63845, relative to untreated control. FIGS. 8B and 8C are images of western blot analysis indicating that ABT-199 and S63845 induce caspase-3 and PARP cleavage, respectively, in MDMs infected with M.tb (in FIGS. 8B and 8C, actin expression is used as a control). These results show that ABT-199 and S63845 induce apoptosis in MDMs, including during M.tb infection.

### Example 8: Combinatorial Therapy with Mcl-1 and Bcl-2 Inhibitors Limits Multi-Drug Resistant M.tb Growth in Infected Human Macrophages

MDMs were infected with a multi-drug resistant strain of M.tb resistant to isoniazid and rifampicin at MOI 1 for 2 hours. The MDMs were washed and incubated with 2% serum +/−10 μM ABT-199 and 10 μM S63845. M.tb CFUs were assessed after 4 days.

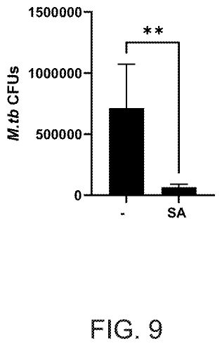

FIG. 9 is a graphical representation of the results of a CFU assay showing that treating human macrophages infected with multi-drug resistant M.tb with a combination of 10 μM S63845 and 10 μM ABT-199 (SA) reduced M.tb growth. Results are mean±standard deviation from 1 experiment, **p<0.01 indicates a significant reduction in M.tb growth with combinatorial treatment, relative to untreated control.

Those skilled in the art will recognize, or be able to ascertain using no more than routine experimentation, many equivalents to the specific embodiments described herein. Such equivalents are intended to be encompassed by the following claims.

What is claimed is:

1. A method of treating an infection caused by an intracellular pathogen, the method comprising administering to a subject in need thereof a therapeutically effective amount of a BCL-2 inhibitor and a therapeutically effective amount of a MCL-1 inhibitor.

2. The method of claim 1, wherein the BCL-2 inhibitor is one or more of venetoclax (ABT-199), ABT-263, S55746, or ABT-737.

3. The method of claim 1, wherein the MCL-1 inhibitor is one or more of S63845, A-1210477, MIM-1, MIK665/S-64315, 483-LM, AZD5991, AMG-176, AMG-397, UMI-77, VU661013, or JKY-5-037.

4. The method of claim 1, wherein the BCL-2 inhibitor is venetoclax and the MCL-1 inhibitor is S63845.

5. The method of claim 1, further comprising administering a first composition comprising from about 2 mg/kg to about 300 mg/kg of the BCL-2 inhibitor.

6. The method of claim 5, further comprising administering a second composition containing from about 2 mg/kg to about 50 mg/kg of the MCL-1 inhibitor.

7. The method of claim 1, wherein the BCL-2 inhibitor is venetoclax and the MCL-1 inhibitor is MIK665/S-64315.

8. The method of claim 1, wherein the BCL-2 inhibitor is venetoclax and the MCL-1 inhibitor is AZD5991.

9. A method of treating a Mycobacterium tuberculosis infection, the method comprising administering to a subject in need thereof a therapeutically effective amount of a BCL-2 inhibitor and a therapeutically effective amount of a MCL-1 inhibitor.

10. The method of claim 9, wherein the BCL-2 inhibitor is one or more of venetoclax (ABT-199), ABT-263, S55746, or ABT-737.

11. The method of claim 9, wherein the MCL-1 inhibitor is one or more of S63845, A-1210477, MIM-1, MIK665/S-64315, 483-LM, AZD5991, AMG-176, AMG-397, UMI-77, VU661013, or JKY-5-037.

12. The method of claim 9, wherein the BCL-2 inhibitor is venetoclax and the MCL-1 inhibitor is S63845.

13. The method of claim 9, wherein the subject has active tuberculosis.

14. The method of claim 13, wherein the method further comprises administering to the subject a therapeutically effective amount of an anti-tuberculosis antibiotic.

15. The method of claim 9, wherein the subject has latent tuberculosis.

16. The method of claim 15, wherein the method further comprises administering to the subject a therapeutically effective amount of an anti-tuberculosis antibiotic.

17. A method of inhibiting growth of Mycobacterium tuberculosis in a granuloma, the method comprising contacting the granuloma with a therapeutically effective amount of a BCL-2 inhibitor and a therapeutically effective amount of a MCL-1 inhibitor.

18. The method of claim 17, wherein the BCL-2 inhibitor comprises venetoclax (ABT-199), ABT-263, S55746, ABT-737, or any combinations thereof.

19. The method of claim 17, wherein the MCL-1 inhibitor comprises S63845, A-1210477, MIM-1, MIK665/S-64315, 483-LM, AZD5991, AMG-176, AMG-397, UMI-77, VU661013, JKY-5-037, or any combinations thereof.

20. The method of claim 17, wherein the method further comprises administering to the subject a therapeutically effective amount of an anti-tuberculosis antibiotic.
